(19) United States
(12) Patent Application Publication
(10) Pub. No.: US 2009/0028786 A1
(43) Pub. Date: Jan. 29, 2009

(54) METHODS AND APPARATUS FOR IMAGING AGENTS FOR DISEASE DETECTION

(75) Inventor: Frederick Randall Grimes, Scottsdale, AZ (US)

Correspondence Address: THE NOBLITT GROUP, PLLC, 4800 NORTH SCOTTSDALE ROAD, SUITE 6000, SCOTTSDALE, AZ, 85251, US

(73) Assignee: RCP Diagnostics, LLC

(21) Appl. No.: 12/177,848

(22) Filed: Jul. 22, 2008

Related U.S. Application Data
(60) Provisional application No. 60/951,395, filed on Jul. 23, 2007.

(51) Int. Cl. A61K 49/04; A61K 51/00; A61K 49/06

(52) U.S. Cl. 424/1.11; 424/9.1; 424/9.4; 424/9.3

(57) ABSTRACT

Methods and apparatus for identifying disease status according to various aspects of the present invention include delivering one or more imaging agents to all or parts of the body for disease observation, screening and/or detection. The methods and apparatus may deliver imaging agents that include any suitable disease-associated compositions, such as riboflavin carrier protein (RCP) and/or RCP modifications and antibodies thereof, labeled with an imaging label. In one embodiment, the imaging agents may be delivered to a selected site or sites in the body and subsequently observed using an imaging method. In another embodiment, the imaging agents may be delivered throughout the body for screening applications. In yet another embodiment, the imaging agents may be delivered throughout the body to detect a reduction and/or increase in cellular metabolism.

### CROSS-REFERENCES TO RELATED APPLICATIONS

This application claims the benefit of U.S. Provisional Patent Application No. 60/951,395, filed 23 Jul. 2007, and incorporates the disclosure of such application by reference.

### BACKGROUND OF INVENTION

Cancer is a pervasive disease affecting significant portions of the world population, as well as most mammalian populations. For example, the American Cancer Society has projected that, for the year 2007, 178,480 women in the United States alone will be diagnosed with breast cancer, and more than 40,000 will succumb to the disease. Similarly, each year, gynecological cancers (ovarian, uterine, and vulvular) account for more than 75,000 new cases, and an additional 27,000 deaths.

The key to successful treatment to most cancers is early detection. Imaging techniques are important diagnostic methods. Standard imaging techniques, however, such as positron emission tomography (PET), have diagnostic limitations. One reason for the current shortcomings of diagnostic imaging techniques may be the radiopharmaceutical or contrast agent commonly used. Currently, the radiopharmaceutical 2-[fluorine-18]-fluoro-2-deoxy-D-glucose (FDG) is used for almost all PET scans in practice. While commonly used, this radiopharmaceutical provides a fairly low signal/noise ratio of approximately 3:1 (i.e., wherein cancerous cells have a radiopharmaceutical uptake rate of 3, and normal tissue cells have a relative uptake rate of 1), and as a result smaller cancer sites may not be detected.

### SUMMARY OF THE INVENTION

Methods and apparatus for identifying disease status according to various aspects of the present invention include delivering one or more imaging agents to all or parts of the body for disease observation, screening and/or detection. The methods and apparatus may deliver imaging agents that include any suitable disease-associated compositions, such as riboflavin carrier protein (RCP) and/or RCP modifications and antibodies thereof, labeled with an imaging label. In one embodiment, the imaging agents may be delivered to a selected site or sites in the body and subsequently observed using an imaging method. In another embodiment, the imaging agents may be delivered throughout the body for screening applications. In yet another embodiment, the imaging agents may be delivered throughout the body to detect a reduction and/or increase in cellular metabolism.

### BRIEF DESCRIPTION OF THE DRAWING FIGURES

A more complete understanding of the present invention may be derived by referring to the detailed description when considered in connection with the following illustrative figures. In the following figures, like reference numbers refer to similar elements and steps.

FIG. 1 is a block diagram of a method for detecting disease using an imaging assent;

FIG. 1 is a block diagram of a method for detecting disease using an imaging agent; and

FIGS. 3A-B illustrates a graphical model relating to the uptake of FDG and RCP in tumors of various sizes as measured by PET.

Elements and steps in the figures are illustrated for simplicity and clarity and have not necessarily been rendered according to any particular sequence. For example, steps that may be performed concurrently or in different order are illustrated in the figures to help to improve understanding of embodiments of the present invention.

### DETAILED DESCRIPTION OF INVENTION

The present invention may be described in terms of functional block components and various processing steps. Such functional blocks may be realized by any number of components configured to perform the specified functions and achieve the various results. For example, the present invention may employ various process steps, compositions, agents, detection techniques, etc. In addition, the present invention may be practiced in conjunction with any number of cancer detection and/or cancer treatment techniques, and the system described is merely one exemplary application for the invention. Further, the present invention may employ any number of conventional techniques for imaging, imaging agent delivery, and the like.

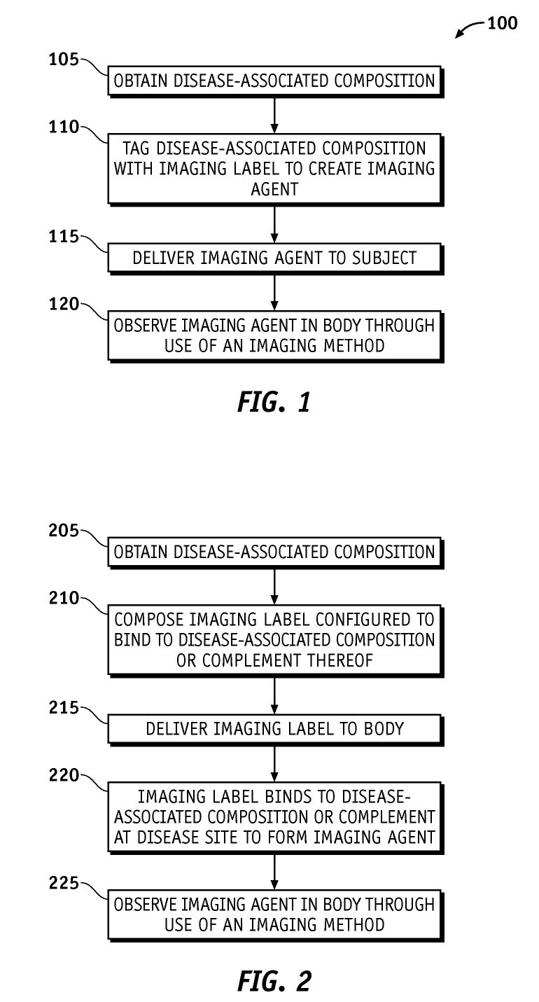

Systems and methods for improved diagnostics using one or more imaging agents according to various aspects of the present invention may be adapted to detect cancer, identify cancer cell metabolic activities, characterize cancer cell growth, indicate early treatment efficacy, detect recurrence of cancer and/or the like. Referring now to FIG. 1, the method for detecting a disease 100 according to the present invention generally includes obtaining a disease-associated composition (105) and tagging the disease-associated composition and/or a complement of the disease-associated composition with an imaging label to create an imaging agent (110). The imaging agent is delivered to the body or parts of the body of a subject (115) and may be observed using an imaging method (120). In alternative embodiments, the disease-associated composition and the imaging label may be separately delivered to the body, wherein the imaging agent is created in vivo.

In one embodiment, the imaging agents may be delivered to a selected site or sites in the body and subsequently observed using an imaging method. In another embodiment, the imaging agents may be delivered throughout the body for screening applications. In yet another embodiment, the imaging agents may be delivered throughout the body to detect a reduction and/or increase in cellular metabolism.

The imaging agent may comprise any disease-associated composition suitable for detection of cancer and/or identification of cancer cell metabolic activities labeled with an imaging label and/or a complement to such disease-associated composition labeled with an imaging label. Effective disease-associated compositions may include any proteins, hormones and/or subunits thereof that facilitate cellular metabolism and/or are the byproducts of cellular metabolism that vary in a linear and/or nonlinear fashion with glucose consumption.

In general, the more non-linear the relationship between glucose metabolism and the production or presence of the disease-associated composition, the better the disease-associated composition will be for the detection of cancer. For example, in one embodiment disease-associated compositions may be present in a factor varying from approximately three-fold to twenty-seven fold higher than glucose taken up (e.g., uptake) during glucose metabolism in cancerous cells as compared to noncancerous cells. This represents a non-linear relationship between the amount of the disease-associated composition present, produced and/or accumulated by cancerous cells as compared to the amount of glucose uptake during glucose metabolism of cancerous cells.

In yet another embodiment, the disease-associated composition may include any suitable antibody, such as monoclonal, polyclonal and artificially synthesized antibody and/or any suitable analog thereto. In yet a further embodiment, the disease-associated composition may include anti-body like molecules that function as synthetic recognition elements.

Effective disease-associated compositions may be produced within the diseased cells and/or elsewhere in the body and delivered to and/or collected by the diseased cells. In one embodiment, disease-associated compositions may be produced and/or accumulated at and/or near the sight of the disease. In another embodiment, disease-associated compositions may be produced and/or accumulated by the disease. In yet another embodiment, disease associated compositions may be delivered at and/or near the sight of the disease.

In one embodiment, disease-associated compositions may include hormone-induced growth and/or development specific proteins and/or protein subunits. In another embodiment, complements to disease-associated compositions may include antibodies to such hormone-induced growth and/or development specific proteins and/or protein subunits.

Effective disease-associated compositions may also include proteins and/or protein subunits that assist in cellular metabolism and or transport of nutrients, such as vitamins and minerals, not capable of being produced by the body, and therefore obtained through diet. Complements to such disease-associated compositions may include antibodies and/or other agents that preferentially bind and/or associate with such proteins and/or antibodies to protein subunits. Examples of such nutrients include riboflavin, vitamin B12 and/or the like.

In one embodiment, effective disease-associated compositions that assist in cellular metabolism comprise riboflavin carrier proteins (RCP). In another embodiment, the disease-associated composition may include modifications of RCP, for example RCP subunits, such as peptide subunits. In another embodiment, effective complements of disease-associated compositions may comprise antibodies to RCP. In yet another embodiment, complements of disease-associated compositions may include antibodies to modifications of RCP, for example antibodies for RCP subunits, such as peptide subunits, antibodies to RCP and/or antibodies to RCP receptors.

RCP is a necessary protein which facilitates transport across the cell membrane of riboflavin, vitamin B12, and/or the like. The absence, blockage and/or binding of RCP may lead to cell death. Fast growing cells, such as embryonic cells and cancer cells, require higher quantities of riboflavin, and hence, higher quantities of RCP. Accordingly, elevated levels of RCP may be found in pregnant women and/or individuals with cancer. Moreover, such elevated levels may also be detected at and/or near the site of embryonic and/or cancer cells. Thus, utilizing RCP as a disease-associated composition in the imaging agent may assist in detection of such fast growing cells.

The imaging label may comprise any substance that may attach to, bind and/or associate with disease-associated compositions and/or complements to disease-associated compositions and is visible to an imaging system. Suitable imaging labels may include radiopharmaceuticals, contrast agents and/or the like. In one embodiment, imaging labels may include radiopharmaceuticals, such as iodine-131, fluorine-18 and/or the like, and may be suitable to use in positron emission tomography (PET). In another embodiment, imaging labels may include contrast agents, such as iodine, barium and/or the like, and may be suitable to use in computerized tomography (CT or CAT) imaging systems. In yet another embodiment, imaging labels may include contrast agents such as paramagnetic, diamagnetic and/or superparamagnetic contrast agents, and may be suitable to use in magnetic resonance imaging (MRI).

In one embodiment, the imaging label attaches and/or binds to the disease-associated composition, such as RCP, to act as the imaging agent. In another embodiment, the imaging label attaches to and/or binds to the disease-associated composition to form an imaging agent prior to delivery to the body.

In another embodiment, the imaging label attaches and/or binds to the disease-associated composition after delivery to the body. In yet another embodiment, the imaging label, once delivered to the body, may attach and/or bind to a disease-associated composition at and/or near the sight of the disease. For example, referring now to FIG. 2, the disease associated composition may be obtained through any suitable method, such as stock and/or through isolation from a blood sample of the patient (205). Thereafter, the imaging label is obtained and configured to bind to the disease-associated composition and/or a complement thereof (210). This imaging label may be subsequently delivered to the body (215) and may travel to and/or come into proximity of the disease-associated composition and/or complements thereof at and/or near the site of a disease and may bind with either and/or both the disease-associated composition and/or a complement thereof (220) to form an imaging agent. The imaging label attaches and/or binds to the disease-associated composition that is produced and/or accumulated by the disease. The imaging agent in the body may be observed through use of an imaging method (225).

In another embodiment, the imaging label attaches and/or binds to the complement of a disease-associated composition, such as antibodies to RCP, to act as the imaging agent. In another embodiment, the imaging label attaches to and/or binds to the complement of a disease-associated composition to form an imaging agent prior to delivery to the body.

Imaging agents may be delivered to the desired site or sites through any suitable mechanism of delivering imaging agents. In one embodiment, the imaging agent is injected into the blood stream of the body. The blood stream may act as a delivery system, carrying the imaging agent to various cells in the body. In another embodiment, the imaging agent may be injected near and/or at a site suspected of comprising cancerous cells. Alternatively, the imaging agent may be delivered via other mechanisms, such as inhalation, ingestion, or through the lymph system.

After delivery, the imaging agents may be tracked to identify areas which quickly and/or slowly accumulate the imaging agent. In the present embodiment, those areas that more quickly absorb the RCP disease-associated composition and/or anti-bodies to the RCP disease-associated composition may correspond to one or more sites of cancerous growth. In one embodiment, the imaging agent, once delivered to the body, may travel to the cancerous cells and may be bound, collected and/or metabolized by these cancerous cells. In another embodiment, the imaging agent, upon delivery to the body, may travel to cancerous cells and at least one of attach to, bind, and/or associate with RCP generated by cancerous cells. In yet another embodiment, the imaging agent, once delivered to the body, may travel to cancerous cells and at least one of attach to, bind, and/or associate with RCP collected by cancerous cells. In yet another embodiment, the imaging agent, once delivered to the body, may travel to and/or be collected by cancerous cells at higher ratios than non-cancerous cells. Differences between cancerous and non-cancerous cells may be observed as differing rates at which imaging agents and/or disease associated compositions travel to, are collected by, attach to, bind to and/or associate with cells in the body. Such differences can be quantified, for example, as true differences (for example, site A concentration−site B or background concentration) or as ratios (for example, site A concentration/site B or background concentration).

The imaging agent may be tracked using a non-invasive technique, such as conventional CT and/or nuclear imaging. The imaging methods may comprise any technique and/or method for identifying, distinguishing, localizing, and/or highlighting cancerous cell growth in the human body through one or more imaging agents. For example, imaging methods may include magnetic resonance imaging (MRI), positron emission tomography (PET), computer tomography (CT), and scintigraphy. In one embodiment, the imaging method may comprise PET, where PET is used to identify cancerous cell growth in any part of the body. In another embodiment, the imaging method may comprise a combination of PET and CT for identification of cancerous growth in any part of the body. In another embodiment, the utilization of PET in combination with imaging agents of the present invention may enhance diagnostic accuracy and/or enable detection of smaller cancer cell sites than what are currently detectable through use of conventional methods.

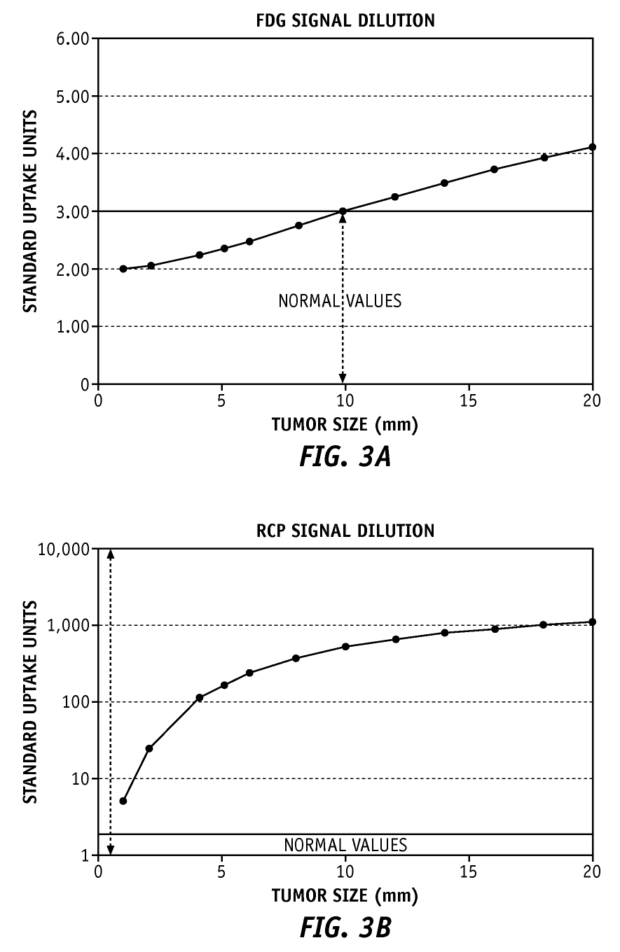

In another embodiment of the present invention, the utilization of PET in combination with imaging agents of the present invention may provide for a higher imaging contrast and/or provide an enhanced signal to noise ratio. For example, the conventional radiopharmaceutical 2-[fluorine-18]-fluoro-2-deoxy-2-D-glucose (FDG) may provide a fairly low relative signal-to-noise ratio of approximately 3:1 (i.e., wherein cancerous cells have a radiopharmaceutical uptake of 3, and normal tissue cells have a relative uptake of 1), and as a result, smaller cancer sites may not be detected. Referring now to FIG. 3A, the cells of normal tissues may have standard uptake units of about 3 or less using PET to detect FDG, whereas tumors of greater than about 10 mm may exhibit standard uptake units of FDG of about 3 or greater. As consequence, the detection of FDG by PET may not be useful for tumors under 10 mm that exhibit standard uptake units of about 3 or less which may coincide with the standard uptake units of FDG of normal cells.

In another embodiment, the utilization of PET in combination with the imaging agents of the present invention may enhance the signal/noise ratio to at least 5:1 where imaging agents attach to and/or associate with and/or bind to RCP collected by the cancerous cells. In yet a further embodiment, the utilization of PET in combination with the imaging agents of the present invention may enhance the signal-to-noise ratio by about at least 1000:1 over the signal-to-noise ratio of detecting FDG by PET where imaging agents attach to and/or associate with and/or bind to RCP generated by the cancerous cells. Referring now to FIG. 3B, the cells of normal tissues may have standard uptake units of about 2 or less using PET to detect RCP and/or complements thereof tagged with an imaging label, whereas tumors as small as about 1 mm may exhibit standard uptake units of RCP of about 8 or greater. As a consequence, utilization of PET as an imaging method in combination with the imaging agent comprising RCP allows for detection of tumors as small as 1 mm or less whereas the tumors must be about 10 mm or larger for successful detection of FDG by PET.

The particular implementations shown and described are illustrative of the invention and its best mode and are not intended to otherwise limit the scope of the present invention in any way. Indeed, for the sake of brevity, conventional processing, data entry, computer systems, and other functional aspects of the system may not be described in detail. Furthermore, the connecting lines shown in the various figures are intended to represent exemplary functional relationships and/or physical couplings between the various elements. Many alternative or additional functional relationships or physical connections may be present in a practical system.

The present invention has been described above with reference to a particular embodiment. However, changes and modifications may be made to the particular embodiment without departing from the scope of the present invention. These and other changes or modifications are intended to be included within the scope of the present invention.

1. A composition for an imaging agent, comprising:
a disease-associated composition; and
an imaging label configured to associate with at least one of the disease-associated composition and a complement of the disease-associated composition.

2. The composition according to claim 1, wherein the disease-associated composition comprises at least one of a protein, a hormone, and sub-unit of the protein, wherein the protein is configured to at least one of facilitate cellular metabolism and comprise a by-product of cellular metabolism.

3. The composition according to claim 2, wherein the protein comprises riboflavin carrier protein.

4. The composition of claim 1, wherein the disease-associated composition is at least one of produced, generated and present at or near the site of the disease in a non-linear relationship to an amount of glucose in at least one of a cancerous cell and a non-cancerous cell.

5. The composition of claim 1, wherein the disease-associated composition is configured to at least one of be present, accumulated, and generated at least one of at and near a site of a disease.

6. The composition of claim 5, wherein the disease comprises a cancer.

7. The composition of claim 1, wherein the complement of the disease-associated composition comprises at least one of an antibody to the disease-associated composition, an antibody to a receptor for the disease-associated composition, and an antibody to a subunit of the disease-associated composition.

8. The composition of claim 1, wherein the imaging label comprises at least one of a radiopharmaceutical and a contrast agent.

9. The composition of claim 8, wherein the radiopharmaceutical is configured for use in conjunction with positron emission tomography.

10. A method for observing a disease in a body comprising:
obtaining an imaging agent comprising a disease-associated composition and an imaging label, wherein the imaging label is configured to at least one of bind and associate with at least one of the disease-associated composition and a complement of the disease-associated composition;
delivering the imaging agent to the body; and
observing the imaging agent in the body.

11. The method of claim 10, wherein the imaging label and the disease-associated composition are delivered to separate points of the body.

12. The method of claim 10, wherein the imaging label is configured to bind to at least one of the disease-associated composition and the complement of the disease associated composition prior to delivery of the imaging agent to the body.

13. The method of claim 10, wherein the imaging agent is observed through an imaging method comprising at least one of positron emission tomography, computerized tomography, magnetic resonance imaging, and scintigraphy.

14. The method of claim 10, wherein the imaging label is configured to attach to at least one of the disease-associated composition and the complement of the disease associated composition near a site of the disease in the body.

15. The method of claim 10, wherein the disease-associated composition is at least one of produced, generated and present at or near the site of the disease in a non-linear relationship to an amount of glucose uptake in at least one of a cancerous cell and a non-cancerous cell.

16. The method of claim 15, wherein the imaging agent comprises the complement of the disease-associated composition, and wherein the imaging agent is configured to bind to at least one of the disease-associated composition.

17. The method of claim 10, wherein:
the imaging agent is observed through positron emission tomography; and
the imaging agent is configured to at least one of bind and associate with riboflavin carrier protein that is at least one of produced at and collected near a site of the disease.

18. The method of claim 17, wherein the riboflavin carrier protein is collected near the site of the disease, and wherein the use of positron emission tomography provides a signal to noise ratio of about 5:1.

19. The method of claim 17, wherein the riboflavin carrier protein is produced at the site of the disease, and wherein the use of positron emission tomography provides a signal to noise ratio of about 1000:1.

20. An imaging agent for cancer detection comprising:
at least one of riboflavin carrier protein, a sub-unit of the riboflavin carrier protein, an antibody to the sub-unit of the riboflavin carrier protein, and an antibody to the riboflavin carrier protein; and
an imaging label configured to bind with the at least one of the riboflavin carrier protein, a sub-unit of the riboflavin carrier protein, an antibody to the sub-unit of the riboflavin carrier protein, and an antibody to the riboflavin carrier protein.
